(19) United States
(12) Patent Application Publication
(10) Pub. No.: US 2007/0270773 A1
(43) Pub. Date: Nov. 22, 2007

(54) Absorbent articles and garments with thermochromic wetness indicator(s)

(76) Inventor: Suzan Delain Mackey, Burns, OR (US)

Correspondence Address: Suzan Delain Mackey, P.O. Box 91, Hines, OR, 97738, US

(21) Appl. No.: 11/434,592

(22) Filed: May 16, 2006

(51) Int. Cl. A61F 13/15

(52) U.S. Cl. 604/361; 604/367

(57) ABSTRACT

The present invention includes absorbent articles and/or garments with thermochromic wetness indicator(s), providing a method of wetness detection with use of products including diapers, incontinent briefs, feminine pads, bandages and other alike absorbent products.

### CROSS REFERENCE TO RELATED APPLICATIONS

Not Applicable

### REFERENCE TO SEQUENCE LISTING A TABLE

Not Applicable

### STATEMENT REGARDING FEDERALLY SPONSORED RESEARCH

Not Applicable

### BACKGROUND OF INVENTION

This invention relates to absorbent articles and/or garments such as diapers, incontinent briefs, feminine pads, bandages and alike absorbent articles and/or garments with thermochromic wetness indicator(s).

Absorbent articles and/or garments that are designed to contain bodily wastes are widely used and needed by both the young and old for many purposes and reasons.

Though these products have advanced technologically greatly through the years, there are problems still unresolved with these products. The most relevant problems are associated with health issues and the negative physical and emotional effects of prolonged exposure to the skin of urine and other bodily excretions. The physical effects include skin irritation, diaper rash, skin sores and infection. Once these symptoms and disorders begin they can be difficult to cure or control because the possibility of continued extended exposure remains.

Another problem is the emotional effects of pain and suffering to an individual, which could include sadness or depression, crying, loss of appetite, insomnia and irritability.

The best cure for the problems is prevention. The need for wetness indicating diapers and/or other absorbent articles and/or garments is real. Many inventors and diaper manufacturing company's have worked hard and have invented many different wetness indicating products, attempting to solve the problems associated with extended exposure of undetected urine, bodily waste and bodily excretions.

This invention is presented to solve the problems of undetected urine and other bodily wastes. In whole, this invention is adaptable to all absorbent articles and/or garments with simple modifications.

U.S. Pat. No. 4,834,733 issued May 30, 1989 to A. Huntoon et al, describes a fluid activated mechanical absorbency gauge which uses a liquid-contractible material which is capable of shrinking upon contact with fluids, thus providing a wetness indicator.

U.S. Pat. No. 6,603,403 issued Aug. 5, 2003 to Jeutter et al, describes a remote wetness signaling system, placed on the wearer which communicates a change of status upon wetness, thus providing a wetness indicator. A similar patent, U.S. Pat. No. 4,484,573 issued Nov. 27, 1984 sounds an alarm upon detection of wetness.

U.S. Pat. No. 6,635,797 issued to Olson et al, on Oct. 21, 2003 describes the use of fluid soluble inks with interactive graphics that changes appearance upon wetness to assist as a training aid for potty training.

U.S. Pat. No. 6,657,099 issued to Underhill et al, on Dec. 2, 2003 describes a sheet for alerting a wearer to urination, using a urine soluble matter incorporated with the sheet that is dissolved by the urine upon urination enabling wetness detection.

U.S. Pat. Nos. 5,197,958 and 5,389,093 issued to Howell, describes reusable diapers with thermally sensitive indicators comprised of thermochromic ink(s) using conventional silk screening techniques as means of incorporation with the reusable diaper. Many other U.S. patents describes the use fluid soluble inks and ph changing indicators as wetness indicators. Most diapers, absorbent articles and/or garments must either be removed to check for a visual change to the inks and ph indicators to detect wetness or require extensive changes to the manufacturing process of such articles and garments to incorporate wetness indicating abilities to existing products, which in turn drives up the manufacturing costs. Other problems encountered with these types of ph indicators and fluid soluble ink wetness indicators is that the wetness indicating effects are wicked away into the absorbent materials making detection difficult or the wetness indicator effects do not have a lasting visual appearance. Adding adhesives and other chemical compounds to ph changing indicators and fluid soluble inks has also been attempted to capture the wetness indicating visual effects for longer periods of time. Other U.S. patents issued describes a viewing window incorporated to the absorbent article or garment to assist in visual wetness detection.

Though progress is being achieved, the present invention offers a new approach.

### SUMMARY OF INVENTION

The invention presented includes absorbent articles and/or garments, such as diapers, incontinent briefs, bandaging, feminine pads and alike absorbent articles and/or garments with thermochromic wetness indicator(s) in the form of thermochromic compound(s) and/or materials(s) including thermochromic leuco dye(s) and/or thermochromic liquid crystal(s).

The object of this invention is to provide products including absorbent articles and/or garments comprised of a liquid-permeable top sheet with an external and an internal side, and a liquid-impermeable back sheet with an external and an internal side and an absorbent pad/core situated at least partially between the internal side of the liquid-permeable top sheet and the internal side of the liquid-impermeable back sheet. The thermochromic wetness indicator(s) may include incorporation with the external side of the liquid-impermeable back sheet and changes color upon the temperature changes to the article and/or garment including the liquid-impermeable back sheet and/or the thermochromic wetness indicator(s), in this case, urine and/or bodily wastes and/or bodily excretions generates the temperature change that activates the thermochromic wetness indicator(s).

Another object of this invention is to provide a quick visual check of the thermo-chromic wetness indicator(s) for early detection of wetness.

Another object of this invention is to eliminate the use of hands to feel or touch for urine and/or other bodily wastes and/or bodily excretions.

Another object of this invention is to prevent disease by spreading bacteria and germs associated with direct/indirect contact and touch transfer of urine and/or other bodily wastes and/or bodily excretions.

Another object of this invention is to prevent skin disorders, medicating and doctor visits associated with extended exposure to skin of urine and/or other bodily wastes and/or bodily excretions. Such skin disorders could include diaper rash, skin irritation, body sores and infection.

Another object of this invention is to save time for caregivers of wearers of absorbent articles and garments. Caretakers, hospitals and other care giving facilities are strapped with a huge amount of responsibilities and expectations that go with their jobs. Checking diapers and other absorbent articles takes time, a quick visual check takes a fraction of a second and the time saved could be better spent providing other important care taking tasks.

Another object of this invention is to reduce waste of absorbent articles and/or garments. The majority of absorbent articles and garments facilitate adhesive fastening features to secure absorbent articles and/or garments in a preferred location consistent with the intended use. Checking the article and/or garment for wetness often requires removal that causes damage, rendering the absorbent article useless and discarded, thus generating unneeded waste and contributing to unwarranted negative environmental impact.

Another object of this invention is to eliminate direct bodily contact to chemical compounds associated with the wetness-indicating embodiment incorporated within the interior of absorbent articles and/or garments.

Another object of this invention is to provide absorbent articles and/or garments that are safe to discard, inexpensive to produce, reasonably priced and is affordable to consumers.

Another object of this invention is to provide a new solution to an old problem using thermochromic compound(s) and/or materials(s) as the wetness indicator(s) embodiment of absorbent articles and/or garments. Most prior art of wetness indicating absorbent articles and/or garments facilitates wetness indicator(s) using various ph compounds, liquid soluble inks and electronic signaling devises.

Another object of this invention is to reduce pain, wound contamination and infection to wearers of bandaging and other medical wound coverings, absorbent articles and/or garments by providing a quick visual check for wetness and saturation of bodily wastes and/or bodily excretions rather than using removal as a method to check for wetness and saturation of bandaging and other absorbent articles and/or garments.

### DETAILED DESCRIPTION OF INVENTION

The present invention includes absorbent articles and/or garments with thermochromic wetness indicator(s). The absorbent articles and/or garments may include diapers, incontinent briefs, feminine pads, bandages and all other suitable absorbent articles and/or garments.

The thermochromic wetness indicator(s) may include incorporation to the exterior surface of absorbent articles and/or garments in practical and visible location(s).

Upon contact of urination and/or other bodily wastes and/or bodily excretions, the temperature of absorbent articles and/or garments and/or the thermochromic wetness indicator(s) increases and the increased temperature change activates a visual color change to the thermochromic compounds(s) and/or material(s) applied as the wetness-indicating embodiment(s).

Any suitable thermochromic wetness indicator(s) may be used with this invention. For example, the thermochromic compound(s) and/or material(s) used with this invention may include various known/unknown thermochromic compound(s) and/or material(s) including thermochromic leuco dye(s) and/or thermochromic liquid crystal(s) or any suitable combination. The thermochromic compound(s) and/or material(s) used as the wetness indicator(s) embodiment of this invention may be of any suitable color or combination of colors both prior to activation and after activation of the wetness indicator(s) occurs. For example, a possible color prior to activation may include tan and after activation occurs the color change may include blue.

Any suitable temperature range may be set for the activation of color change to the thermochromic wetness indicator(s) and could include the range of known human bodily temperatures. For example, the activation temperature range for color change of the wetness indicator(s) could include 95 F to 104 F, however due to the unknown insulation factors and/or RH factors and/or uses of future absorbent articles and/or garments developed with this invention, the actual activation temperature range of the wetness indicator(s) will be determined upon product development.

The method of application of the thermochromic compounds(s) and/or material(s) including thermochromic leuco dye(s) and/or liquid crystal(s) to the absorbent articles and/or garments as the wetness indicator(s) could include printing, painting, dying, staining and/or melting and could be manually applied and/or mechanically applied. Furthermore, the thermochromic compound(s) and/or materials(s) used as the wetness indicator(s) embodiment(s) of this invention may include inks, paints, powders, vinyl and/or any other thermochromic compound(s) and/or material(s) not currently available for use. Specific application method(s) and thermochromic material(s) and/or compound(s) are to be predetermined at the time of product development.

The graphic form of application of the thermochromic wetness indicator(s) may include any suitable shapes, figures, characters, animals, numbers, letters, people, planes, trains and/or automobiles, flowers, trees, insects, and/or any combination of such, either entirely of thermochromic compound(s) and/or material(s) including thermochromic leuco dye(s) and/or thermochromic liquid crystal(s) or combined with other non thermochromic compound(s) and/or materials(s) and/or medium(s) of any form and/or of any graphic content as described above.

An example of the present invention is comprised of a liquid-permeable top sheet with an external and an internal side, a liquid-impermeable back sheet with an external and internal side, and a absorbent core/pad situated at least partially between the internal sides of the liquid-permeable top sheet and the liquid-impermeable back sheet, and a thermochromic wetness indicator(s) may include incorporation to the exterior side of the liquid-impermeable back sheet, furthermore the present invention offers a large range and variety of uses with many products including diapers, incontinent briefs, feminine pads, bandages and any other suitable products and/or uses.

Providing this invention is an important step forward in personal care products. Early detection of urine and/or other bodily wastes and/or bodily excretions is a priceless asset for providing a healthy and clean environment for both the wearers and the caretakers of the wearers of absorbents articles and/or garments alike. Controlling and preventing diaper rash, skin irritation, body sores and infection through early detection of wetness is a win-win situation for all.

Although the absorbent articles and/or garments are described to include thermochromic wetness indicator(s), those articles and/or garments may include any suitable wetness indicator(s), including any suitable wetness indicator(s) that change color based, at least in part, on temperature, moisture-content, and/or other suitable parameters. Additionally, although absorbent articles and/or garments and features of absorbent articles and/or garments have been shown and described with reference to the foregoing operational principles and preferred embodiments, those skilled in the art will find apparent that various changes in form and detail may be made. Inventions may be embodied in various combinations and sub-combinations of features, functions, elements, and/or properties, which may be claimed in a future application. Such claims are regarded as included within the subject matter of the present disclosure.

### DESCRIPTION OF DRAWING

The drawing provided with the present invention is only a visual tool to simply illustrate the basic concept of this invention. The provided drawing is only an example of a possible use of the thermochromic wetness indicator(s) incorporated with absorbent articles and/or garments as described herein. It should be considered generic and not of a particular absorbent article or garment. FIG. 1, sheet 1 of 1 shows an incontinent brief (1) including an opening (2) and a thermochromic wetness indicator (3).

FIGS. 1, 1: Represents a front external view of an incontinent brief.

FIGS. 1, 2: Represents the view of the opening of an incontinent brief.

FIGS. 1, 3: Represents a thermochromic wetness indicator.

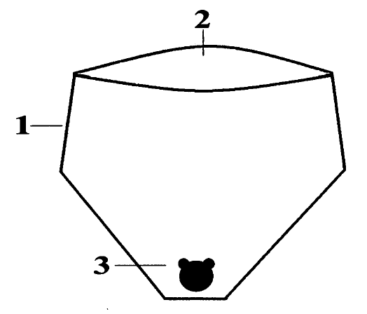

I claim my invention is:

1. Absorbent articles and/or garments with thermochromic wetness indicator(s), with embodiments including a liquid-permeable top sheet with an internal and external side, a liquid-impermeable back sheet with an interior and exterior side, with an absorbent core/pad situated at least partially between the internal side of said top sheet and internal side of said back sheet.

2. As per claim 1, the thermochromic wetness indicator(s) is comprised of thermochromic compound(s) and/or material(s) including thermochromic leuco dye(s) and/or thermochromic liquid crystal(s).

3. As per claim 1, the thermochromic wetness indicator(s) may include incorporation with the exterior surface of the liquid-impermeable back sheet.

4. As per claim 3, the graphic form of the thermochromic wetness indicator(s) may include any suitable shapes, figures, characters, animals, numbers, letters, people, planes, trains and/or automobiles, flowers, trees, insects, and/or any combination of such, either entirely of thermochromic compound(s) and/or material(s) including thermochromic leuco dye(s) and/or liquid crystal(s) or combined with other non thermochromic compound(s) and/or material(s) and/or medium(s) in any suitable form(s) and/or of any graphic content as described above, and will be predetermined upon product development.

5. As per claim 4, the thermochromic wetness indicator(s) may include being incorporated including incorporation of the external side of the liquid-impermeable back sheet by means of printing, painting, gluing, staining, dyeing, melting or by means of any other predetermined method of affixation, attachment and/or application, either manually and/or mechanically applied.

6. As per claim 3, the thermochromic wetness indicator(s) may be located in any singular or multiple position(s) and/or location(s) including the exterior side of the liquid-impermeable back and will be predetermined upon product development.

7. As per claim 3, the thermochromic wetness indicator(s) may be of any single or multiple color or color combinations both prior to activation and after activation occurs.

8. As per claim 7, the thermochromic wetness indicator(s) activation occurs upon exposure to temperature change generated by urine, and/or bodily wastes and/or bodily excretions, either with direct or indirect contact of the absorbent article and/or garment and/or the thermochromic wetness indicator(s).

9. As per claim 1, the absorbent article and/or garment embodiment may include a liquid-permeable top sheet with an external and an internal side, a liquid-impermeable back sheet with an external and internal side, and an absorbent core/pad situated at least partially between the internal sides of the liquid-permeable top sheet and the liquid-impermeable back sheet, and a thermochromic wetness indicator(s) may include incorporation to the exterior side of the liquid-impermeable back sheet, however the present invention offers a large range and variety of uses and products including diapers, incontinent briefs, feminine pads, bandages and any other suitable products and/or uses presently not discovered and/or described herein and the exact embodiment will be predetermined upon further product development.
